(19) United States
(12) Patent Application Publication
(10) Pub. No.: US 2021/0038660 A1
(43) Pub. Date: Feb. 11, 2021

(54) ENHANCED VIRAL DELIVERY FORMULATION

(71) Applicant: Ascend Biopharmaceuticals Ltd, South Melbourne (AU)

(72) Inventors: Clement Leong, South Melbourne (AU); Geoffrey Allan Pietersz, South Melbourne (AU)

(73) Assignee: Ascend Biopharmaceuticals Ltd, South Melbourne (AU)

(21) Appl. No.: 16/936,025

(22) Filed: Jul. 22, 2020

Related U.S. Application Data
(63) Continuation of application No. PCT/AU2019/050037, filed on Jan. 22, 2019.

(30) Foreign Application Priority Data
Jan. 23, 2018 (AU) 2018900204

(51) Int. Cl. A61K 35/761; A61K 38/21; A61K 47/69

(52) U.S. Cl. CPC A61K 35/761; A61K 47/6903; A61K 38/217

(57) ABSTRACT

The present invention relates generally to recombinant adenoviral pharmaceutical formulations. More particularly, the present invention relates to SiO₂-gel-based controlled release recombinant adenoviral pharmaceutical formulations.

### FIELD OF THE INVENTION

The present invention relates generally to recombinant adenoviral pharmaceutical formulations. More particularly, the present invention relates to SiO₂-gel-based controlled release recombinant adenoviral pharmaceutical formulations.

### BACKGROUND OF THE INVENTION

Non-replicative, recombinant adenoviruses have gained widespread use in a number of therapeutic areas such as gene therapy and cancer therapy. However, a number of challenges remain for the effective application of non-replicative recombinant adenoviruses in the clinic, including stabilisation of infectivity at elevated (non-cryogenic) temperatures, control of their release and expression profile for long-term treatments, and minimisation of an immune response following administration.

Thus, there is an ongoing need for controlled release recombinant, non-replicative adenovirus-based pharmaceutical formulations for optimised therapeutic efficacy and safety.

### SUMMARY OF THE INVENTION

The present inventors have surprisingly found that one or more non-replicative recombinant adenoviruses formulated with SiO₂ hydrogel particles, in addition to stabilising adenovirus infectivity, yields enhanced expression of an encoded biotherapeutic agent.

Accordingly, in one aspect provided herein is a pharmaceutical composition comprising:

(i) one or more non-replicative recombinant adenoviruses for expression of one or more biotherapeutic agents;

(ii) SiO2 matrix gel particles; wherein the one or more non-replicative recombinant adenoviruses are interspersed in the SiO₂ matrix hydrogel, and wherein the pharmaceutical composition does not comprise a chemotherapeutic agent.

In a particularly preferred embodiment, a therapeutically effective dose of the one or more non-replicative recombinant adenoviruses in the pharmaceutical composition is lower than a therapeutically effective dose of the same non-replicative recombinant adenoviruses not formulated in the pharmaceutical composition.

In some embodiments the one or more biotherapeutic agents are selected from the group consisting of: cytokines, chemokine, chemokine agonist, chemokine antagonist, chemokine receptor antagonist, costimulatory molecules, checkpoint inhibitors, metalloproteinase inhibitors, matrix metalloproteinase (MMPs) inhibitors, tissue inhibitors of metalloproteinases (TIMPs) and antibodies. In other embodiments, the one or more biotherapeutic agents are selected from the group consisting of interferon gamma, interferon alpha, interleukin 12, interleukin 15, CD40L, Ox40L, 4-1BB, ICOS-L, LIGHT, CD70, TGF-beta, Hyaluronidase (PH20), an CD200 antagonist, an PD1 antagonist, an PDL1 antagonist, an CTLA-4 antagonist, an LAG3 antagonist, an CD27 agonist, a TGF-beta antagonist, leukocyte immunoglobulin-like receptor antagonists and a LAIR-1 antagonist. In some preferred embodiments one or more of the CD200 antagonist, the PD1 antagonist, the PDL1 antagonist, the CTLA-4 antagonist, the LAG3 antagonist, the TGF-beta antagonist, the leukocyte immunoglobulin-like receptor antagonist or the LAIR-1 antagonist is an antibody.

In some embodiments, the one or more biotherapeutic agents comprise a chemokine. In some embodiments, the one or more biotherapeutic agents comprise a costimulatory molecule. In some embodiments, the one or more biotherapeutic, agents comprise a checkpoint inhibitor. In some embodiments, the one or more biotherapeutic agents comprise a metalloproteinase inhibitor. In some embodiments, the one or more biotherapeutic agents comprise a matrix metalloproteinase (MMTs) inhibitor.

In other embodiments, the one or more encoded biotherapeutic agents to be expressed comprise a cytokine. In some preferred embodiments the cytokine is interferon gamma. In some preferred embodiments, the non-replicative recombinant adenovirus is ASN-002 which encodes interferon gamma.

In an embodiment, expression of at least one of the biotherapeutic agents is higher in vivo when compared to a corresponding pharmaceutical composition lacking the SiO₂ matrix hydrogel. In an embodiment, expression of at least one of the biotherapeutic agents is about 2 fold to about 10 fold, or about 2 fold to about 5 fold, or at least 2 fold, or at least 4 fold, higher in vivo when compared to a corresponding pharmaceutical composition lacking the SiO₂ matrix hydrogel.

In some embodiments, the one or more non-replicative recombinant adenoviruses comprises a first and a second non-replicative recombinant adenoviruses each of which is for expression of a different biotherapeutic agent. In some preferred embodiments, one of the non-replicative recombinant adenovirus is encodes a cytokine as one of the one or more biotherapeutic agents. In some embodiments one of the non-replicative recombinant adenovirus encodes CD40L or an CD27 agonist as one of the one or more biotherapeutic agents.

In some embodiments, the SiO₂ matrix hydrogel comprises tetraethyl orthosilicate (TEOS). In some embodiments the SiO₂ matrix hydrogel comprises water and TEOS in a final molar ratio of about 5:1 to about 4,000:1 or about 5:1 to about 1,000:1. In some preferred embodiments the final molar ratio of water to TEOS is about 400:1.

In some embodiments, the pharmaceutical composition, when administered, releases the one or more non-replicative adenoviruses in vivo over a period of about one day to about 48 hours or about 1 day to about 30 days.

In some embodiments, the one or more non-replicative adenoviruses retain about 50% to about 75%, or at least about 50%, of their infectivity after contact of the pharmaceutical composition with a cell culture medium at 37° C. for 24 hours.

In some embodiments, the one or more non-replicative recombinant adenoviruses retain at least about 50% to about 75%, or at least about 50%, of their infectivity when the pharmaceutical composition is maintained at about 4° C. for about 12 months to about 24 months.

In some embodiments, the pharmaceutical composition is a depot formulation.

In some embodiments, the pharmaceutical compositions comprises one or more pharmaceutically acceptable excipients.

In some embodiments, the one or more pharmaceutically acceptable excipients comprise one or more polyols. In some embodiments the one or more polyols are selected from the group consisting of sucrose, mannitol, ethanol, trehalose, sorbitol, glycerol and polyethylene glycol. In some preferred embodiments the one or more polyols comprise sucrose and ethanol. In other preferred embodiments the one or more polyols comprise glycerol and sucrose. In some preferred embodiments the pharmaceutically acceptable excipients comprise glycerol., sucrose, phosphate buffer, NaCl and MgCl₂.

In some embodiments, the one or more pharmaceutically acceptable excipients further comprise one or more detergents. In some embodiments the one or more detergents are selected from the group consisting of Polyoxyethylene (20) sorbitan monooleate (Polysorbate 80), Polyethylene glycol sorbitan monopalmitate (Polysorbate 40), Polyoxyethylene (20) sorbitan monolaurate (Polysorbate 20) and 3-[(3-Cholamidopropyl)dimethylammonio]-1-propanesulfonate. In some embodiments the one or more detergents comprise Polysorbate 80.

In some embodiments, the one or more pharmaceutically acceptable excipients further comprise one or more antioxidants. In some embodiments the one or more antioxidants comprise histidine, triethanolamine (TEOA), citrate and ethylenediaminetetraacetic acid (EDTA). In some preferred embodiments the one or more antioxidants comprise EDTA and histidine. In some preferred embodiments the one or more pharmaceutically acceptable excipients comprise sucrose, ethanol, EDTA, histidine, polysorbate 80, NaCl and MgCl₂.

In some embodiments the pharmaceutical composition comprises about 1×10¹⁰ viral particles/Pal to about 5×10¹² viral particles/ml.

In a related aspect, provided herein is a method for treating a subject suffering from a disease, comprising administering to the subject a therapeutically effective amount of any of the foregoing pharmaceutical compositions.

In an embodiment, the disease is cancer. In some embodiments, the subject is suffering from a cancer selected from the group consisting of basal cell carcinoma, squamous cell carcinoma, colorectal cancer, ovarian cancer, breast cancer, gastric cancer and pancreatic cancer. in some embodiments the subject is suffering from a basal cell carcinoma or squamous cell carcinoma. In some embodiments the subject is suffering from a cancer comprising one or more lesions or tumours. In some embodiments the pharmaceutical composition is injected into at least one of the one or more lesions or tumours.

In a related aspect provided herein is the use of any of the above-mentioned pharmaceutical compositions in the manufacture of a medicament for treating a disease.

In a further aspect, the present invention provides for the use of

(i) one or more non-replicative recombinant adenoviruses for expression of one or more biotherapeutic agents; and

(ii) SiO2 matrix hydrogel:

in the manufacture of a medicament for treating a subject suffering from a disease, wherein the one or more non-replicative recombinant adenoviruses are interspersed in the SiO2 matrix hydrogel, and wherein the pharmaceutical composition does not comprise a chemotherapeutic agent.

In a further related aspect is the use of a pharmaceutical composition provided herein for use in the treatment of a disease.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (e.g. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—Stability of biological activity of ASN-002 in cell culture medium at 37° C. A scatter plot showing the release of IFN-γ from H-1299 cells following infection with ASN-002 at various ratios of viral particles/cell (vp/cell) and following various incubation times at 37° C. (n=3).

FIG. 2—Biological activity of ASN-002 in R400 sols at different pH after 24 hours at 37° C. A scatter plot showing the release of IFN-γ from H-1299 cells following infection with ASN-002-R400 SiO₂ gel matrix formulations (made with varying pH) at various vp/cell ratios, and following incubation times at 37° C. for 24 hours (n=3).

FIG. 3—Biological activity of ASN-002 in sol after 24 hours at 37° C. A bar graph summary of data from FIG. 3. The data are is shown for an infection ratio of 3.3 virus particles/cell. The R400 (pH 6 and 7) preparation of virus produce significantly more IFN-γ on infection compared to initial T=0.

FIG. 4—Comparison of biological activity of ASN-002 versus ASN-002 in depot formulations. Line graphs showing expression of IFN-γ measured after incubation of non-formulated ASN-002 and ASN-002 formulations R5-400 and R150-400 in H-1299 cells for 24 hr. Note that infection is expressed in cells/virus particles on x-axis.

FIG. 5—Stability of biological activity of intact ASN-002 after thawing and 12 days of storage at 4° C. A scatter plot comparing the infectivity of ASN-002 (unformulated) following a 12 day storage period at 4° C. versus the infectivity of freshly thawed ASN-002.

FIG. 6—Comparison of biological activity between encapsulated ASN-002 and intact ASN-002. A scatter plot comparing the infectivity of ASN-002 R150-400 and R5-400 formulations following a 7 day storage period at 4° C. versus the infectivity of control ASN-002 (“placebo”-ASN-002 with only SiO₂ microparticles).

FIG. 7—Release of ASN-002 in dissolution test based on biological activity. A bar graph summary of the results shown in FIG. 5 and FIG. 6 for a VP/cell ratio of 3.3.

FIG. 8—Dissolution of ASN-002 R150-400 depot formulation. A scatter plot showing the infectivity of the R150-400 formulation, at various dilutions, following 2 hour, 3 hour and 5 hour incubation times to allow release of ASN-002 in culture medium.

FIG. 9—IE-HPLC analysis of virus particle release on dissolution of ASN-002 R150-400 depot formulation. A summary table of viral particle release from the ASN-002 R150-400 formulation following incubation in a buffered Tris solution for 1, 2 and 4 hours.

FIG. 10—Analysis of viral particle release and infections titer of R100-400. A summary table of viral particle release from the ASN-002 R100-400 formulation following incubation in a buffered Tris solution for 1, 2 and 4 hours.

FIG. 11—In vitro dissolution test of R100-400 depot formulation: Cumulative release of viral particles and their infectious titer. A scatter plot of viral particle release and infectivity from the ASN-002 R100-400 formulation following incubation in a buffered Tris solution for 1, 2 and 4 hours.

FIG. 12—In vitro dissolution test of R100-400 depot formulation: IFN gamma assay of dissolution samples. Line graphs showing expression of IFN-γ measured after incubation of non-formulated ASN-002 and ASN-002 formulations R5-400 and R100-400 in H 1299 cells for 24 hr.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, cell biology, viral vector construction, gene therapy, molecular genetics, cancer biology, cancer therapy, immunology, pharmacology, protein chemistry, and biochemistry).

Unless otherwise indicated, the cell culture and immunological techniques utilized in the present invention are standard procedures, well known to those skilled in the art. Such techniques are described and explained throughout the literature in sources such as, J. Perbal, A Practical Guide to Molecular Cloning, John Wiley and. Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et at. (editors) Current Protocols in Immunology, John Wiley & Sons (including all updates until present).

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements., integers or steps.

As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “recombinant adenovirus”, as used herein, refers to any adenovirus that is genetically modified by experimental intervention.

The term “biotherapeutic agent”, as used herein, refers to any biologically-active molecule, such as one that can be used to treat a cancer, which can be expressed from a recombinant non-replicative adenovirus. Such biotherapeutic agents include by way of example only, a cytokine, an antibody, a receptor body, an RNAi, a miRNA, or an sgRNA.

The term “chemotherapeutic agent” refers to a class of small molecules that is cytostatic and/or cytotoxic to cancer cells. For the avoidance of any doubt, the biotherapeutic agent may be a chemotherapeutic agent. However, a pharmaceutical composition of the invention will not comprise a chemotherapeutic agent not expressed by the virus. In other words, the virus will only express the biotherapeutic agent when released from the matrix and it infects a cell. Thus, the pharmaceutical composition does riot comprise chemotherapeutic agents per se added to the formulation through the intervention of man.

As used herein, the term the “expression of at least one of the biotherapeutic agents is higher in vivo when compared to a corresponding pharmaceutical composition lacking the SiO₂ matrix hydrogel” means that the composition of the invention results in higher levels of expression of the biotherapeutic agent.

As used herein, the term “subject” can be any animal. In one example, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the mammal is a human.

The term “antibody” as referred to herein, includes polyclonal antibodies, monoclonal antibodies, bispecific antibodies, fusion diabodies, triabodies, heteroconjugate antibodies, chimeric antibodies including intact molecules as well as fragments thereof and other antibody-like molecules. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain.

The terms “effective amount” or “therapeutically effective dose”, as used herein, refer to a sufficient amount of at least one recombinant virus that will relieve to some extent one or more of the symptoms of the disease or condition being treated (e.g., a cancer). The result can be reduction and/or alleviation of the signs, symptoms, or causes of a disease, or any other desired alteration of a biological system.

The terms “treating” or “treatment,” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a pharmaceutical composition) or (ii) instruct a third party to treat a subject according to a claimed method. Also encompassed within the meaning of the term “treating” or “treatment” are prevention of relapse or reduction of the disease to be treated, e.g., by administering a therapeutic at a sufficiently early phase of disease to prevent or slow its progression.

### Controlled Release Non-Replicative Recombinant Adenovirus Pharmaceutical Compositions

Provided herein are controlled release non-replicative recombinant adenovirus pharmaceutical compositions. Controlled release refers to the release of adenoviruses from a dosage form in which they are incorporated according to a desired profile over an extended period of time. Controlled release profiles include, for example, sustained release, prolonged release, pulsatile release and delayed release profiles. In contrast to immediate release compositions, controlled release compositions allow delivery of one or more non-replicative recombinant adenoviruses to a subject over an extended period of time according to a predetermined profile. Such release rates can provide therapeutically effective levels of adenovirus-mediated gene expression for an extended period of time and thereby provide a longer period of therapeutic response while minimizing side effects as compared to conventional rapid release forms. In addition such compositions are less likely to induce an immune response than recombinant adenovirus administered in a standard formulation. Such longer periods of response provide for many benefits that are not achieved with the corresponding short acting, immediate release preparations.

Pharmaceutical compositions of the invention comprise (i) one or more non-replicative recombinant adenoviruses for expression of one or more biotherapeutic agents and (ii) SiO₂ matrix hydrogel; wherein the one or more non-replicative recombinant adenoviruses are interspersed in the SiO₂ matrix hydrogel; wherein the pharmaceutical composition does not comprise a chemotherapeutic agent. In an embodiment, the therapeutically effective dose of the one or more non-replicative recombinant adenoviruses in the pharmaceutical composition is lower, such as 10% to 90%, or 10% to 50% lower, or about 5 fold to about 10 fold less, than a therapeutically effective dose of the same non-replicative recombinant adenoviruses not formulated in the pharmaceutical composition.

The controlled release pharmaceutical compositions provided herein allows the release profile of one or more non-replicative recombinant adenoviruses within the formulation to be customised so that release of one or more of these occurs over a preferred time interval. In some embodiments, the one or more non-replicative recombinant adenoviruses are released over a time period ranging from about one hour to about five weeks, e.g., 2 hours, 3 hours, 4 hours, 6 hours, 8 hours, 12 hours, 18 hours, 24 hours, 2 days, 3 days, 5 days, 1 week, 10 days, 2 weeks, 18 days, 3 weeks, 4 weeks, or another period from about one hour to about five weeks. In other embodiments in vivo release occurs over a period of about 3 days to about 30 days. In some embodiments the one or more non-replicative recombinant adenoviruses are released over a time period ranging from about one hour to about 48 hours, or about 18 hours to about 36 hours.

In some embodiments, the controlled release profile has a release rate higher at the beginning of the release period following administration and then decreases over time (first order release kinetics). In other embodiments, the release rate progressively increases over the release period following administration. In preferred embodiments, the release profile remains relatively constant over the entire release period following administration until all of the one or more non-replicative recombinant adenoviruses are released (zero order release kinetics).

In preferred embodiments, the release profile of the one or more non-replicative recombinant adenoviruses upon administration of the pharmaceutical composition is adapted to avoid induction of more than a moderate immune response in the human subject. in some embodiments, the release rate of the one or more non-replicative recombinant adenoviruses is about 5% of the total dose/day to about 100% of the total dose per day, e.g., 6%, 8%, 10%, 15%, 20%, 25%, 30%, 40%, 60%, 70%, 80%, 90%, 95%, or another percentage of the total dose per day from about 0,5% to about 100% per day.

### SiO₂ Matrix Hydrogel

In some embodiments, the SiO₂ matrix hydrogel is a bioresorbable sol-gel derived Tetraethyl orthosilicate (AKA “tetrathoxysilane” or “TEOS”) Si (OC₂H₅)₄ matrix gel (“SiO₂ matrix gel”) as described in WO2005082781 and WO2007135224. This technology has been commercialised by DelSiTech Ltd (Turku, Finland).

For example, the SiO₂ matrix gel sol-gel is prepared by the sol-gel process wherein the SiO₂ matrix gel is prepared from a sol comprising SiO₂ that has turned to a gel. Sol-gel derived SiO₂ is typically prepared from alkoxides or inorganic silicates that via hydrolysis form a sol that contains either partly hydrolysed silica species or fully hydrolysed silicic acid. Consequent, condensation reactions of SiOH containing species lead to formation of larger silica species with increasing amount of siloxane bonds. Furthermore, the species aggregate, form nanosized particles and/or larger aggregates until a gel is formed. In the form of a gel, the solid state dominates, but the system still contains varying amounts of liquids and the material is typically soft and viscoelastic before drying and hard and brittle if it is extensively dried. In the form of a sol, liquid state dominates, but the system contains varying amounts of solid phase(s) and the material is still flowable. The time from when the SiO₂ sol is prepared until the sol turns to a gel is referred to as sol ageing time. Spontaneous drying typically occurs when the sol is aged so that the system allows evaporation in ambient conditions. Generation of the controlled release pharmaceutical composition is achieved by adding to the sol, before gel formation, the desired titres the of one or more non-replicative recombinant adenoviruses for expression of one or more biotherapeutic agents.

Release rates of the active agents in SiO₂ gel-based controlled release pharmaceutical compositions can be adjusted as needed. Generally the maximum dissolution rate of the SiO₂ gel matrix and release rate of the active agents occurs for SiO2 hydrogels having a final molar ratio of water to alkoxide of about 2, with ratios lower or higher than this resulting in slower dissolution and release rates. Further, it should also be noted that large amounts of active agent comprised within the SiO₂ gel matrix increases dissolution of the matrix and the release rate(s) of the active agents.

In some embodiments the rate of adenoviral release (rate of dissolution) observed for a pharmaceutical composition described herein occurs at approximately ten times the rate in vitro than it does in vivo.

In exemplary, non-limiting embodiments, the pH of a water and tetraethyl orthosilicate (TEOS) mixture at an initial molar ratio of about 100:1 to 150:1 is adjusted to pH 2 with hydrochloric acid and vigorously stirred at room temperature for 25 min. The pH of the sol is then adjusted to the desired pH (6, 6.5 or 7) by adding 0.1 M NaOH. The sol is cooled in an ice-water bath and the desired amount of recombinant adenovirus is added (e.g. about 5×10¹⁰ vp/ml to 5×10¹¹ vp/ml). The sol is then diluted with water so that the final water:TEOS ratio is 400:1.

Placebo microparticles (also referred to herein as “secondary microparticles”) generated as described below are added to the sol in a ratio 0.5 g placebo microparticies per 1 ml. The suspension is then allowed to gel and used to fill syringes or the syringes can be filled with the suspension and allowed to gel in a rotator (3 days 24° C. and 9 days at 4° C.).

In exemplary embodiments, placebo microparticles (also referred to herein as “secondary microparticles”) are generated by using water:TEOS at a ratio of 5:1 with HCl as catalyst (pH 2). The resulting sol is then diluted with ethanol, and the pH is adjusted to 6.3. The diluted sol is spray dried using a spray dryer. In some embodiments the water:TEOS ratio of secondary microparticies is from about 2:1 to about 20:1, e.g., about 4:1, 5:1, 6:1, 7:1, 8:1, 911, 10:1, 12:1, 14:1, 15:1, 18:1, or another ration of water to TEOS from about 2:1 to about 20:1.

In some embodiments the SiO₂ matrix hydrogel in the pharmaceutical composition comprise water and TEOS in a final molar ratio of about 5:1 to about 4,000:1, e.g., 10:1, 25:1, 50:1, 75:1, 100:1, 150:1, 200:1, 300:1, 400:1, 500:1, 750:1, 1,000:1, 2,0001, 3,000:1, or another final molar ratio of water to TEOS from about 50:1 to about 700:1, or about 5:1 to about 1,000:1. In some preferred embodiments the final molar ratio of water to TEOS is about 400:1.

An additional advantage of the pharmaceutical compositions described herein is the stabilisation of adenoviral infectivity at elevated temperatures. In some embodiments of the pharmaceutical compositions described herein the one or more non-replicative recombinant adenoviruses retain at least about 50% to about 75% of their infectivity after contact of the pharmaceutical composition with a mammalian cell culture medium at 37° C. for 24 hours. In other embodiments the one or more non-replicative recombinant adenoviruses retain at least about 50% to about 75% of their infectivity when the pharmaceutical composition is maintained at 4° C. for about 12 months to 24 months, e.g., 13 months, 14 month, 15, months, 16 months, 17 months, 18 months, 20 months, 21 months, 22 months, 23 months, or another period from about 12 months to 24 months.

### Non-Replicative, Recombinant Adenoviruses

Adenovirus genomes are linear, 36-Kb double-stranded DNA (dsDNA) molecules containing multiple, heavily spliced transcripts. At either end of the genome are inverted terminal repeats (ITRs). Genes are divided into early (E1-4) and late (L1-L5) transcripts. Advantages of adenoviral gene transfer include the ability to infect a wide variety of cell types, including non-dividing cells, a mid-sized genome, ease of manipulation, high infectivity and they can be grown to high titers (Volpers and Kochanek, 2004; Wilson, 1996). Furthermore, adenoviral infection of host cells does not result in chromosomal integration because adenoviral DNA remains episomal, without potential genotoxicity associated with other viral vectors. Adenoviruses also are structurally stable (Marienfeld et al., 1999) and no genome rearrangement has been detected after extensive amplification (Parks et al 1997; Bett et al 1993).

Non-replicative, recombinant adenoviruses are generally deficient in at least one gene function required for viral replication, thereby resulting in a “non-repticative” adenoviral vector. As used herein, the term “non-replicative” refers to a recombinant adenovirus that comprises an adenoviral genome that lacks at least one replication-essential gene function (i.e., such that the adenoviral vector does not replicate in host cells). A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (e.g., propagation) and are encoded by, for example the adenoviral early regions (e.g., the E1, E2, and E4 regions), late regions (e.g., the L1-L5 regions), genes involved in viral packaging (e.g., the IVa2 gene), and virus associated RNAs (e.g., VA-RNA-1 and/or VA-RNA-2).

In some embodiments, the non-replicative recombinant adenovirus comprises an adenoviral genome deficient in at least one replication-essential gene function of one or more regions of the adenoviral genome. In preferred embodiments, the non-replicative recombinant adenovirus is deficient in at least one essential gene function of the E1 region of the adenoviral genome required for viral replication. In addition to a deficiency in the E1 region, the recombinant adenovirus can also have a mutation in the major late promoter (MLP). The mutation in the MLP can be in any of the MLP control elements such that it alters the responsiveness of the promoter, as discussed in WO 00/00628. More preferably, in some embodiments, the non-replicative recombinant adenovirus is deficient in at least one essential gene function of the E1 region and at least part of the E3 region (e.g., an Xba I deletion of the E3 region). With respect to the E1 region, the non-replicative recombinant adenovirus can be deficient in at least part of the E1a region and at least part of the E1b region. For example, the non-replicative recombinant adenovirus can comprise a deletion of the entire E1 region and part of the E3 region of the adenoviral genome (for example nucleotides 355 to 3,511 and 28,593 to 30,470). Examples of methods of preparing non-replicative recombinant adenoviruses are described in U.S. Pat. Nos. 5,837,511, 5,851,806, 5,994,106, 6,579,522, US 2001/0043922, US 2002/0004040, US 2002/0031831, US 2002/0110545, WO 95/34671, WO 97/12986, and WO 97/21826.

Examples of suitable promoters for driving expression of biotherapeutic agents from a recombinant adenovirus formulated in the pharmaceutical composition described herein include, but are not limited to, constitutive promoters such as, CMV, CAG, EF-1-I, HSV1-TK, SV40, ∝-actin and PGK promoters. In other embodiments, a promoter is an inducible promoters, such as those containing TET-operator elements. In certain embodiments, target-selective promoters are used to drive expression of biotherapeutic agents in specific cell types or specifically in cancer cells. Examples of suitable cancer/cell type-selective promoters useful for the methods described herein include, but are not limited to, the erb 2 promoter (breast cancer), the carcinoembryonic antigen promoter (colorectal cancer), the urokinase-type plasminogen activator receptor promoter (colorectal cancer), the tyrosinase promoter (melanoma), the melacortin receptor (melanoma); the human telomerase reverse transcriptase (hTERT) promoter (multiple cancers), the RAS-related nuclear protein promoter (multiple cancers), the breast cancer metastasis suppressor 1 promoter (multiple cancers), the Rad51C promoter (multiple cancers) and the minichromosome maintenance complex component 5 promoter (multiple cancers).

In some embodiments the one or more recombinant adenoviruses do not comprise an expression cassette for β-galactosidase or a luciferase. In other embodiments the one or more recombinant adenoviruses do not comprise an expression cassette for a reporter protein.

In some embodiments, where two or more proteins are to be expressed from a recombinant virus, the one or more recombinant adenoviruses contains an expression cassette encoding a polycistronic mRNA (a “polycistronic expression cassette”), which, upon translation gives rise to independent polypeptides comprising different amino acid. sequences or functionalities. In some embodiments, a polycistronic expression cassette encodes a “polyprotein” comprising multiple polypeptide sequences that are separated by encoded by a picornavirus, e.g., a foot-and-mouth disease virus (FMDV) viral 2A peptide sequence. The 2A peptide sequence acts co-translationally, by preventing the formation of a normal peptide bond between the conserved glycine and last proline, resulting in ribosome skipping to the next codon, and the nascent peptide cleaving between the Gly and Pro. After cleavage, the short 2A peptide remains fused to the C-terminus of the “upstream” protein., while the proline is added to the N-terminus of the “downstream” protein. which during translation allow cleavage of the nascent polypeptide sequence into separate polypeptides (see, e.g., Trichas et al. (2008).

In other embodiments, a polycistronic expression cassette may incorporate one or more internal ribosomal entry site (IRES) sequences between open reading frames incorporated into the polycistronic expression cassette. IRES sequences and their use are known in the art as exemplified in, Martinez-Sales (1999).

In some embodiments, a recombinant adenovirus used in the method has targeted tropism, e.g., tropism for a particular cell type as reviewed in Yamamoto et al. (2017) and Yoon et al. (2016). Suitable targeting moieties, to be incorporated into a recombinant viral capsid surface, include ligands that bind to cell surface receptors that are overexpressed by cancer cells. For example, CXCL12 has been used to retarget adenovirus vectors to cancer cells via the CXCR4 chemokine receptor (Bhatia et al., 2016).

### Expressed Biotherapeutic Agents

Biotherapeutic agents suitable for the pharmaceutical compositions described herein include biological molecules that can be genetically encoded and expressed by the one or more non-replicative recombinant adenoviruses in such pharmaceutical compositions. Thus, biotherapeutic agents may include peptides, proteins, as well as non-coding RNAs such as short hairpin RNAs (shRNAs), microRNAs (miRNAs), miRNA inhibitors, antisense RNAs, or any combination thereof. Preferably, the biotherapeutic agents to be expressed in humans have highest sequence identity to a human homolog. In some embodiments the sequence of the biotherapeutic agent to be expressed in humans is at least about 80% identical to the human homolog, e.g., 82%, 85%, 88%, 90%, 92%, 95%, 97%, 99%, or another percent identical to the human homolog sequence ranging from about 80% to 100% identical to the human homolog sequence.

In some preferred embodiments a therapeutically effective dose of the one or more non-replicative recombinant adenoviruses in the pharmaceutical composition is lower than a therapeutically effective dose of the same type of non-replicative recombinant adenoviruses not formulated in the pharmaceutical composition. In some embodiments the therapeutically effective dose of the one or more non-replicative recombinant adenoviruses is about 20% to about 80% lower than a therapeutically effective dose of the same type of non-replicative recombinant adenoviruses not formulated in the pharmaceutical composition, e.g., about 25%, 30%, 40%, 50%, 360%, 70%, 80%, or another percentage lower dose from about 20% to about 80% lower.

In some embodiments, the one or more biotherapeutic agents are selected from the group consisting of: cytokines, chemokine, chemokine antagonist., chemokine receptor antagonist, costimulatory molecules, checkpoint inhibitors, metalloproteinase inhibitors, matrix metalloproteinase (MMPs) inhibitors, tissue inhibitors of metalloproteinases (TIMPs) and antibodies.

In other embodiments the one or more biotherapeutic agents are selected from the group consisting of interferon gamma, interferon alpha, interleukin 12, interleukin 15, CD40L (GenBank NP_000065.1), 0x40L (GenBank NP_003317.1), 4-1BB (GenBank AAA53133.1), ICOS-L (GenBank AAH64637.1), LIGHT (GenBank CAG46652.1), CD70 (GenBank AAH00725.1), TGF-beta, Hyaluronidase (PH20; GenBank AAH26163.1), an CD200 antagonist, an PD1 antagonist, an PDL1 antagonist, an CTLA-4 antagonist, an LAG3 antagonist, a TGF-beta antagonist, leukocyte immunoglobulin-like receptor antagonist and a LAIR-1 antagonist. In some preferred embodiments one or more of the CD200 antagonist, the PD1 antagonist, the PDL1 antagonist, the CTLA-4 antagonist, the LAG3 antagonist, the TGF-beta antagonist, the leukocyte immunoglobulin-like receptor antagonist or the LAIR-1 antagonist is an antibody.

In an embodiment, the chemokine antagonist is an CxCL12 (SDF1) antagonist.

In an embodiment, the chemokine receptor antagonist is a CxCR4 antagonist.

In some preferred embodiments the one or more biotherapeutic agents comprise a chemokine. In other preferred embodiments the one or more biotherapeutic agents comprise a costimulatory molecule. In other preferred embodiments the one or more biotherapeutic agents comprise a checkpoint inhibitor.

In some preferred embodiments the one or more biotherapeutic agents comprise a cytokine. In one particularly preferred embodiment the cytokine is interferon gamma. In one embodiment, wherein the cytokine is interferon gamma, one of the one or more non-replicative recombinant adenoviruses in the pharmaceutical composition is ASN-002 (also known as Tg1042) (Urosevic, 2007; Liu et al., 2004; Dummer et al., 2004 and 2010; Accart et al., 2013; Khammari et al., 2015; Dreno et al., 2014; Hillman et al., 2004).

Other suitable cytokines to be expressed include, but are not limited to, interferon gamma, interferon alpha, B-cell activating factor (BAFF), TL1, TNT alpha, TRAIL, lymphotoxin alpha, lymphotoxin beta, OX-40 ligand, LIGHT (also known as tumor necrosis factor superfamily member 14), FAS-ligand, 4-1BB ligand, RANK ligand, CD30 ligand, CD40 ligand, glucocorticoid-induced TNFR-related protein ligand (GITRL), or any combination thereof.

In some embodiments of any of the pharmaceutical compositions described herein the one or more non-replicative recombinant adenoviruses comprise first and second non-replicative recombinant adenoviruses each of which is fir expression of a different biotherapeutic agent. In some embodiments one of the non-replicative recombinant adenoviruses encodes CD40L or an CD27 agonist as one of the one or more biotherapeutic agents. In some embodiments one of the non-replicative recombinant adenovinises encodes a cytokine as one of the one or more biotherapeutic agents.

In some preferred embodiments, the sequence of a biotherapeutic agent to be expressed comprises the sequence of the human homolog of (e.g., the amino acid. sequence of human IFN gamma or the human nucleic acid sequence encoding human IFN gamma).

Other suitable types of protein biotherapeutic agents to be expressed include, but are not limited to a cytokine, a protein regulating apoptotic cell death, a protein regulating necroptotic cell death, a protein regulating parthanatos cell death, or a protein regulating autophagic cell death, or an agonist which binds a cell receptor and. activates cell death by apoptosis necroptosis, parthanatos autophagic cell death, or any combination thereof.

In some embodiments, the biotherapeutic agent to be expressed is an agonist antibody to the FAS receptor (FasR), e.g., a scFv antibody such as the “E09” scFv antibody described in Chodorge et al. (2012).

in other embodiments, a biotherapeutic agent to be expressed by a recombinant virus used in the treatment method includes a non-coding RNA. Such non-coding RNAs include short hairpin RNAs (shRNAs) to effect RNA interference, microRNAs (miRNAs), miRNA inhibitors, anti sense RNAs including antisense RNAs against miRNAs (e.g., “miRNA sponges” as described in Ebert et al. 2007).

The terms “RNA interference”, “gene silencing” and related phrases refers generally to a process in which a double-stranded RNA molecule reduces the expression of a nucleic acid sequence with which the double-stranded RNA molecule shares substantial or total homology. However, it has more recently been shown that RNA interference can be achieved using non-RNA double stranded molecules (see, for example, US 20070004667).

By “shRNA” or “short-hairpin RNA” is meant an RNA molecule where less than about 50 nucleotides, preferably about 19 to about 23 nucleotides, is base paired with a complementary sequence located on the same RNA molecule and where said sequence and complementary sequence are separated by an unpaired region of at. least about 4 to about 15 nucleotides which forms a single-stranded loop above the stem structure created by the two regions of base complementarity.

Included shRNAs are dual or hi-finger and multi-finger hairpin dsRNAs, in which the RNA molecule comprises two or more of such stem-loop structures separated by single-stranded spacer regions.

In some embodiments, a non-coding RNA to be expressed as a biotherapeutic agent is an shRNA against a cancer target. Suitable shRNA cancer targets include, but are not limited to, Cyclin D1 (GenBank BC023620.2), Class III α-tubulin (GenBank NM_006086), Receptor for activated C-kinase 1 (RACK1; GenBank NM006098); Ras homolog gene family member A (RI-10A.; GenBank BC001360), Mitogen-activated protein kinase-activated protein kinase 5 (MAPKAPKS; GenBank NM003668); Growth differentiation factor-11 (GDF11; GenBank AF028333), Engrailed 1 (EN1; GenBank NM_001426.3) and Microphthalmia-associated transcription factor (MITF; GenBank NM_000248).

In other embodiments a non-coding RNA to be expressed is a miRNA. Suitable examples of a miRNA to be expressed in a treatment method described herein include, but are not limited to, mir-491, mir-133a, mir-204, let 7 miRNA, mir-24, mir-15a, mir-16, mir-26a, mir-148b, mir-199a-3p, mir-512, mir-874a, or any combination thereof. Suitable examples of suitable miRNA targets for suppression in cancer cells, e.g., by expression of an miRNA sponge, include, but are not limited to mir-223, mir-211, mir-9, mir-17-92, mir-103, mir-1060, mir-107 mir-155, mir-21, mir-128, or any combination thereof.

In further embodiments a, non-coding RNA to be expressed is a single guide RNA (“sgRNA”), which can be used for CRISPR-based targeted disruption of a gene in combination with a programmable nuclease such as Cas9 nuclease, which may be co-expressed by a single recombinant adenovirus or expressed separately from a second recombinant adenovirus. Suitable examples of sgRNA include, but are not limited to, sgRNAs against CTLA4 or PD-1, PDL1, CTLA-4, LAG3, TFG-heta receptor, or LAIR1. sgRNA sequences are commercially available, e.g., from Thermo Fisher Scientific.

In certain embodiments a recombinant virus to be used in the treatment method expresses at least two biotherapeutic agents, two proteins; a non-coding RNA and a protein; or two non-coding RNAs.

In some preferred embodiments, the two biotherapeutic agents to be expressed include a cytokine and a protein selected from among MLKL, SMAC, the N-terminal tetrapeptide (AVPI) of SMAC (Guo et al., 2002), BAX, DAI, cyclic (MP-AMP synthase (cGAS; GenBank NP_612450.2) and RIPK3.

### Pharmaceutically Acceptable Excipients and Administration

Non-replicative adenoviruses described herein can be formulated as a pharmaceutical composition suitable for administration to a subject. In some embodiments the pharmaceutical compositions described herein comprise one or more pharmaceutically acceptable excipients, Such excipients may provide the additional benefit of stabilising the infectivity of the one or more recombinant, non-replicative adenoviruses present in the pharmaceutical compositions described herein. The choice of excipient will be determined, in part, by the particular site to which the composition is to be administered and the particular method used to administer the composition. Depending upon the particular route of administration, a variety of acceptable excipients, known in the art may be used, as for example described in Remington's Pharmaceutical Sciences (Mack Publishing Co. N.J. USA, 1991).

Suitable pharmaceutical compositions include aqueous and non-aqueous solutions, hydrogels, isotonic sterile solutions, which can contain anti-oxidants, butlers, bacteriostats, and solutes that render the composition isotonic with the bodily fluid at the site of administration, and aqueous and non-aqueous sterile suspensions that can include suspending agents, solubilizers, thickening agents, stabilizers, and preservatives.

The pharmaceutical compositions described herein can be presented in unit-dose or multi-dose sealed containers, such as ampules and vials, and can be stored in a freeze-dried (lyophilized) condition requiring only the addition of the sterile carrier, for example, water, immediately prior to use. Extemporaneous solutions and suspensions can be prepared, for example, from sterile powders, granules, and tablets. In some embodiments, the non-replicative recombinant adenovirus is administered in a pharmaceutical composition formulated to protect and/or stabilize the adenovirus from damage prior to administration. For example, the pharmaceutical composition can be formulated to reduce loss of the non-replicative recombinant adenovirus on devices used to prepare, store, or administer the expression vector, such as glassware, syringes, pellets, slow-release devices, pumps, or needles.

Pharmaceutical composition can also be formulated to decrease light sensitivity and/or temperature sensitivity of the non-replicative recombinant adenovirus. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a pharmaceutical composition can extend the shelflife of the non-replicative recombinant adenovirus, facilitate administration, and increase the efficiency of gene transfer. In this regard, a pharmaceutical composition can be formulated to enhance transduction efficiency.

In some preferred embodiments the pharmaceutical composition is prepared as a formulation suitable for injection. In some preferred embodiments the injectable formulation is a depot formulation.

Formulations suitable for intralesional, intramuscular, subcutaneous, or intravenous injection may include physiologically acceptable sterile aqueous or non-aqueous solutions, dispersions, suspensions or emulsions.

In some embodiments pharmaceutically acceptable excipients present in the pharmaceutical compositions described herein include one or more polyols. In some embodiments the one or more polyols are selected from the group consisting of sucrose, mannitol, ethanol, trehalose, sorbitol, glycerol and polyethylene glycol. In other embodiments the one or more polyols comprise sucrose and ethanol. In some preferred embodiments a pharmaceutical composition described herein comprises about 0.5% ethanol (v/v) and about 5% sucrose (w/v). In other preferred embodiments the one or more polyols comprise glycerol and sucrose. In some preferred embodiments the pharmaceutical composition described herein comprises about 10% glycerol (w/v) and about 2% sucrose (w/v).

In one exemplary embodiment the pharmaceutically acceptable excipients present in the pharmaceutical compositions described herein include glycerol, sucrose, phosphate buffer, NaCl and MgCl₂.

In some embodiments any of the above-mentioned pharmaceutical compositions also include one or more detergents. In some embodiments the one or more detergents are selected from the group consisting of Polyoxyethylene (20) sorbitan monooleate (Polysorbate 80), Polyethylene glycol sorbitan monopalmitate (Polysorbate 40), Polyoxyethylene (20) sorbitan monolaurate (Polysorbate 20) and 3-[(3-Cholamidopropyl)dimethylammonio]-1-propanesulfonate. In some embodiments the one or more detergents include Polysorbate 80.

In some embodiments any of the above-mentioned pharmaceutical compositions also include one or more antioxidants. In some embodiments the one or more antioxidants are selected from the group consisting of histidine, triethanolamine (TEOA), citrate and ethylenediaminetetraacetic acid (EDTA). in some preferred. embodiments the one or more antioxidants comprise EDTA and histidine.

In an embodiment, the composition comprises one or more or all of protamine, poly-L-lysine and polyethyleneimine.

In some preferred embodiments the excipients in the pharmaceutical compositions described herein comprise sucrose, ethanol, EDTA, histidine, polysorbate 80, NaCl and MgCl₂.

The pharmaceutical compositions described herein may be formulated in aqueous solutions, preferably in physiologically compatible buffers such as Hank's solution, Ringer's solution, or physiological saline buffer.

In one exemplary embodiment the pharmaceutical composition comprises 10 mM Tris, 75 mM NaCl, 5% (w/v) sucrose, 0.020% (w/v) polysorbate 80, 1 mM MgCl2, 100 μmM EDTA, 0.5% (v/v) EtOH, 10 mM His, pH 7.4.

In another exemplary embodiment, the pharmaceutical composition comprises 10% Glycerol, 10-20 nM Phosphate buffer, pH 8 or 14 mM Tris/HCl (pH 7.80), 100 mM NaCl mM, MgCl2, 2% sucrose and optionally 0.015% (w/v) polysorbate 80.

In a further exemplary embodiment the pharmaceutical composition comprises 5% sucrose or 5% Trehalose, 5% human serum albumin or 1% PEG3500 in 10 mM Tris (pH8.2), 0.15 M NaCl and 1 mM MgCl2.

In yet another exemplary embodiment the pharmaceutical composition comprises 5% sucrose, 1% glycine, 1 mM MgCl2, 10 mM Tris (pH 7.8) and 0.05% Tween 80. In a further exemplary embodiment the pharmaceutical composition comprises 5% sucrose, 1% glycine, 1 mM MgCl2, 10 mM Tris, 8% F-127 (CAS number9003-11-6).

In some embodiments the pharmaceutical composition is prepared as a formulation suitable for topical administration. Formulations suitable for topical administration are well known to those of skill in the art. Such formulations are suitable for application to, for example, a subject's eye, skin, or lesion. The use of patches, cortical shields (see, U.S. Pat. No. 5,185,152), and ophthalmic solutions (see U.S. Pat. No. 5,710,182) and ointments, e.g., eye drops, is also within the skill in the art. The pharmaceutical formulation can also be administered non-invasively using a needleless injection device, such as the Biojector 2000 Needle-Free Injection Management System® available from Bioect, Inc.

### Dosing

The person of ordinary skill in the art will appreciate that a suitable therapeutically effective dose of the one or more recombinant, non-replicative adenoviruses provided in the pharmaceutical compositions described herein will depend upon factors such as the particular biotherapeutic agent to be expressed, the cells transduction characteristics of the recombinant adenovirus, the stage of the disease, the characteristics of the subject or host in need of treatment (e.g., weight) and the properties of the particular type of disease to be treated, but can nevertheless be determined in a manner known in the art according to the particular circumstances surrounding the case, including, e.g., the route of administration, the disease being treated, and the subject being treated. The desired dose may conveniently be presented in a single dose or as divided doses administered simultaneously (or over a short period of time) or at appropriate intervals, for example as two, three, four or more sub-doses per day.

It will be understood by those skilled in the art that the dosage regimen to treat the disease for which relief is sought, can he modified in accordance with a variety of factors. These factors include the specific combination of therapeutic agents being used, the disease type and stage from which the subject suffers, as well as the age, weight, sex, diet and medical condition of the subject.

The pharmaceutical compositions described herein may comprise a range of viral titers, expressed as a 50% tissue culture infective dose (TCID₅₀)/ml and/or viral particles (vp)/ml, depending on a number of considerations including the condition to be treated, the subject to be treated, a desired release rate and the desired treatment period per dose. In some embodiments the pharmaceutical compositions described herein have a titer of about 1×10⁹ TCID₅₀/ml to about 3×10¹⁰ TCID₅₀/ml, e.g., 1.5×10⁹ TCID₅₀/ml, 1.8×10⁹ TCID₅₀/ml, 2.0×10⁹ TCID₅₀/ml, 3.0×10⁹ TCID₅₀/ml, 4.0×10⁹ TCID₅₀/ml, 5.0×10⁹ TCID₅₀/ml, 5.5×10⁹ TCID₅₀/ml, 6.0×10⁹ TCID₅₀/ml, 6.5×10⁹ TCID₅₀/ml, 7.0×10⁹ TCID₅₀/ml, 7.5×10⁹ TCID₅₀/ml, 8.0×10⁹ TCID₅₀/ml, 8.5×10⁹ TCID₅₀/ml, 9.0×10⁹ TCID₅₀/ml, 1.0×10¹⁰ TCID50/ml, 1.5×10¹⁰ TCID₅₀/ml, 2.0×10¹⁰ TCID₅₀/ml, 2.5×10¹⁰ TCID₅₀/ml, or another TCID₅₀/ml value from about 1×10⁹ TCID₅₀/ml to about 3×10¹⁰ TCID₅₀/ml, In some preferred embodiments, the TCID₅₀/ml is about 4×10⁹ TCID₅₀/ml to 8×10⁹ TCID₅₀/ml.

In some embodiments the equivalence of vp/TCID₅₀ is approximately 20 to 100 vp/TCID₅₀. Accordingly, in some embodiments the pharmaceutical compositions described herein have a titer of about 2×10¹⁰ vp/ml to about 3×10¹² vp/ml, e.g., 2×10¹⁰ vp/ml, 3×10¹⁰ vp/ml, 4×10¹⁰ vp/ml, 5×10¹⁰ vp/ml, 6×10¹⁰ vp/ml, 7×10¹⁰ vp/ml, 8×10¹⁰ vp/ml, 9×10¹⁰ vp/ml, 1×10¹⁰ vp/ml, 2×10¹¹ vp/ml, 3×10¹¹ vp/ml, 4×10¹¹ vp/ml, 5×10¹¹ vp/ml, 6×10¹¹ vp/ml, 7×10¹¹ vp/ml, 8×10¹¹ vp/ml, 9×10¹¹ vp/ml, 1×10¹² vp/ml, 2×10¹² vp/ml, or another titer from about 2×10¹⁰ vp/ml to about 3×10¹² vp/ml. In some preferred embodiments the titer is from about 3×10¹⁰ vp/ml. to about 8×10¹¹ vp/ml. In other preferred embodiments the titer of the pharmaceutical composition is about 3×10¹⁰ viral particles/ml to about 5×10¹² viral particles/ml.

A particular advantage of the claimed invention is that due to the composition of the invention resulting in enhanced transgene expression allows the practitioner to use less virus. Not only does this save on manufacturing costs but also patient's generally prefer to be administered with as low as does possible a recombinant virus. Lower doses can also assist in reducing unwanted side effects of the agent(s). In an embodiment, the therapeutically effective dose of the one or more non-replicative recombinant adenoviruses in the pharmaceutical composition is lower, such as 10% to 90%, or 10% to 50% lower, or about 5 to about 10 fold less, than a therapeutically effective dose of the same non-replicative recombinant adenoviruses not formulated in the pharmaceutical composition.

In some preferred embodiments, administration of the pharmaceutical compositions described herein recombinant virus is by an intralesional route of administration, in some embodiments, the administered intralesional dose of one or more recombinant, non-replicative adenoviruses present in the pharmaceutical compositions described herein is from about 1×10⁷ vp/lesion to about 1×10¹² vp/lesion, 2×10⁷, 3×10⁷, 4×10⁷, 5×10⁷, 6×10⁷, 8×10⁷, 1×10⁸, 1.5×10⁸, 2×10⁸, 3×10⁸, 4×10⁸, 6×10⁸, 8×10⁸, 9×10⁸, 1×10⁹, 2×10⁹, 3×10⁹, 4×10⁹, 5×10⁹, 6×10⁹, 8×10⁹, 1×10¹⁰, 2×10¹⁰, 3×10¹⁰, 4×10¹⁰, 5×10¹⁰, 6×10¹⁰, 8×10¹⁰, 9×10¹⁰, 1×10¹¹, 2×10¹¹, 3×10¹¹, 4×10¹¹, 5×10¹¹, 6×10¹¹, 8×10¹¹, 9×10¹¹, or another number of vp/lesion from about 1×10⁷ vp/lesion to about 1×10¹² infectious particles/lesion. In some preferred embodiments, the intralesional viral dose ranges from about 1×10⁸ vp/lesion to about 1×10¹¹ vp/lesion.

In other embodiments, the one or more recombinant non-replicative adenoviruses expressing one or more biotherapeutic agents are administered by a systemic, intraperitoneal, or intrapleural route. In some embodiments, the systemic, intraperitoneal, or intrapleural dose of the one or more recombinant, non-replicative adenoviruses is from about 1×10⁸ vp to about 1×10¹³ vp per administration, e.g., 2×10⁸, 3×10⁸, 4×10⁸, 5×10⁸, 6×10⁸, 8×10⁸, 1×10⁹, 1.5×10⁹, 2×10⁹, 3×10⁹, 4×10⁹, 6×10⁹, 8×10⁹, 9×10⁹, 1×10¹⁰, 2×10¹⁰, 3×10¹⁰, 4×10¹⁰, 5×10¹⁰, 6×10¹⁰, 8×10¹⁰, 1×10¹¹, 2×10¹¹, 3×10¹¹, 4×10¹¹, 5×10¹¹, 6×10¹¹, 8×10¹¹, 9×10¹¹, 1×10¹², 1.5×10¹², 2×10¹², 3×10¹², 4×10¹², 5×10¹², 6×10¹², 8×10¹², 9×10¹², or another number of vp per administration from about 1×10⁸ vp to about 1×10¹³ vp. In preferred embodiments the dose is about 1×10⁹ to about 1×10¹² vp.

In other embodiments, where administration of the one or more recombinant non-replicative adenoviruses is intralesional, the total aggregate dose of recombinant viral particles per treatment cycle ranges front about 1×10⁸ vp/lesion to about 1×10¹³ vp/lesion, e.g., 2×10⁸, 3×10⁸, 4×10⁸, 5×10⁸, 6×10⁸, 8×10⁸, 1×10⁹, 1.5×10⁹, 2×10⁹, 3×10⁹, 4×10⁹, 6×10⁹, 8×10⁹, 9×10⁹, 1×10¹⁰, 2×10¹⁰, 3×10¹⁰, 4×10¹⁰, 5×10¹⁰, 6×10¹⁰, 8×10¹⁰, 1×10¹¹, 2×10¹¹, 3×10¹¹, 4×10¹⁰, 5×10¹¹, 6×10¹¹, 8×10¹¹, 9×10¹¹, 1×10¹², 1.5×10¹², 2×10¹², 3×12¹², 4×10¹², 5×10¹², 6×10¹², 8×10¹², 9×10¹² or another number of total vp per treatment cycle from about 1×10⁸ vp/lesion to about 1×10¹³ vp/lesion.

In some embodiments, where administration of the one or more recombinant non-replicative adenoviruses is by systemic, intraperitoneal, or intrapleural administration, the total aggregate viral dose per treatment cycle for a recombinant virus is about 1×10⁹ vp to about 1×10¹⁴ vp per treatment cycle, e.g., 2×10⁹, 3×10⁹, 4×10⁹, 5×10⁹, 6×10⁹, 8×10⁹, 1×10¹⁰, 2×10¹⁰, 3×10¹⁰, 4×10¹⁰, 5×10¹⁰, 6×10¹⁰, 8×10¹⁰, 9×10¹⁰, 1×10¹¹, 1.5×10¹¹, 2×10¹¹, 3×10¹¹, 4×10¹¹, 6×10¹¹, 8×10¹¹, 9×10¹¹, 1×10¹², 2×10¹², 3×10¹², 4×10¹², 5×10¹², 6×10¹², 8×10¹², 9×10¹², 1×10¹³, 2×10¹⁰, 3×10¹³, 4×10¹³, 5×10¹³, 6×10¹³, 8×10¹³, 9×10¹³ or another number of total vp per treatment cycle from about 1×10⁹ vp to about 1×10¹⁴ particles.

In an embodiment, the virus is ASN-002 and the effective dose, namely the amount of virus in the composition administered to a site (for example lesion) in the subject is less than about 2×10¹¹ vp, or about 10¹⁰ to about 7×10¹⁰ vp.

In some embodiments, a subject to be treated is administered treatment with a pharmaceutical composition as described herein over multiple treatment cycles. The number of treatment cycles may range from 1 to 7, e.g., 2, 3, 4, 5, 6 or another number of treatment cycles from 1 to 7. Where a subject is treated over multiple administration cycles, the total aggregate dose recombinant virus per treatment cycle may be varied among different treatment cycles.

In some embodiments, where the subject to be treated is suffering from basal cell carcinoma, the subject a treatment cycle comprises 2-3 administrations in a single week. In other embodiments, where the subject to be treated is suffering from basal cell carcinoma, a treatment cycle comprise 2-3 administrations in two weeks.

In a case where a subject's status does improve, upon reliable medical advice, doses being administered may be temporarily reduced or temporarily suspended for a certain length of time (e.g., a “treatment holiday”). The length of the treatment holiday can vary between 2 days and 1 year, including by way of example only, 2 days, 3 days, 4 days, 5 days, 6 days, 7 days, 10 days, 12 days, 15 days, 20 days, 28 days, 35 days, 50 days, or 60 days. The viral dose reduction during a treatment holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95% or 100%.

### Methods for Treating Disease

Also provided herein is a method for treating a subject (e.g., a human subject) suffering from a disease, comprising administering to the subject a therapeutically effective amount of a pharmaceutical composition described herein. Examples of diseases which can be treated, depending on the biotherapeutic agent include, but are not limited to, cancer, cystic fibrosis, fibrosis, wound healing, autoimmune diseases, infections, ocular diseases, HIV, psychiatric diseases, neurological diseases, coronary diseases and muscular diseases. Examples of the use of adenoviruses to treat such diseases is described in Liu et al. (2011), Rosenfield et al. (1992), McElrath et al. (2008), Han et al. (1999), Lesch (1999), Hermens and Verhaagen (1998), Feldman et al. (19%), Petrof (1998), Dorai et al. (1999), Inci et al. (1999), Mincheff et al. (2000), Blackwell et al. (1999), Stewart et al. (1999), Matra et al. (1999) and Vanderkwaak and Alvarez (1999).

### Cancers

Cancers that can be treated by administration of the pharmaceutical compositions provided herein include, but are not limited to, acute lymphoblastic leukemia, acute myeloid leukemia, adrenocortical carcinoma, anal cancer, astrocytoma, basal cell carcinoma, bladder cancer, bone tumor, breast cancer, Burkitt's lymphoma, cervical cancer, chondrosarcoma, colorectal cancer, cutaneous T-cell lymphoma, endometrial cancer, esophageal cancer, Ewing's sarcoma, intraocular melanoma, retinoblastoma, gallbladder cancer, gastric (stomach) cancer, hairy cell leukemia, head and neck cancer, Hepatocellular (liver) cancer, Hodgkin lymphoma, Kaposi sarcoma, kidney cancer (renal cell cancer), laryngeal cancer oral cancer, Liposarcoma, lung cancer, lymphomas, bone/osteosarcoma, melanoma, Merkel cell cancer, myeloma, neuroblastoma, ovarian cancer, pancreatic cancer, parathyroid cancer, prostate cancer, rectal cancer, renal cell carcinoma (kidney cancer), retinoblastoma, Ewing family of tumors, uterine cancer, skin cancer (non-melanoma), skin carcinoma, small intestine cancer, soft tissue sarcoma, squamous cell carcinoma, squamous neck cancer, stomach cancer, testicular cancer, throat cancer, thyroid cancer, thyroid cancer and uterine cancer. In some preferred embodiments, the subject to be treated is suffering from a cancer selected from among colorectal cancer, basal cell carcinoma, breast cancer, colorectal cancer, ovarian cancer, cervical cancer, melanoma, non-melanoma skin cancer, gastric cancer and pancreatic cancer. In some embodiments, the cancer to be treated include one or more tumours to be treated.

Symptoms, diagnostic tests and prognostic tests for various types of cancers are known in the art. See, e.g., the website of the National Comprehensive Cancer Network (nccn.org/professionals/physician_gls/f_guidelines.asp).

In some embodiments, the subject to be treated by the methods described herein is a subject identified as suffering from a cancer that is refractory or resistant to treatment with chemotherapeutic agents. In some embodiments, the subject to be treated is a subject that was previously treated, unsuccessfully, for the cancer by administration of one or more chemotherapeutic agents. In other embodiments, the treatment methods described herein also include determining, prior to the treatment, whether a subject is suffering from a cancer that is refractory or resistant to treatment with chemotherapeutic agents. In some embodiments the treatment methods described herein specifically exclude treatment of a subject suffering from cancer with a pharmaceutical composition described herein in combination with a chemotherapeutic agent (e.g., a nucleotide analogue chemotherapeutic agent).

### EXAMPLE 1

### Preparation and In Vitro Testing of Injectable ASN-002 Adenovirus Vector Silica Hydrogel Depot Formulations

### Materials and Methods

ASN-002 (also known as Tg1042) is a genetically modified replication-defective adenovirus type 5 based vector in which the E1 and E3 regions have been deleted and the virus engineered to express interferon-gamma (IFN-γ). More specifically, the genome comprises
the left end of the human adenovirus type 5 (Ad5) comprising the left ITR, the encapsidation signal, and the enhancer of the adenovirus E1a promoter;
in place of the deleted E1 region, the passenger gene comprises:
   the immediate early enhancer/promoter region from the human Cytomegalovirus (pCMV);
   a chimeric intron (Int) made of the donor site from the human β-globin intron 1 and the acceptor and branch point from a murine IgG gene to increase the overall transcriptional efficiency of the recombinant gene;
   the cDNA sequence coding for IFNγ whose primary structure is identical to the structure that can be deduced from the RNA sequence described in Genbank (reference: V00543). The IFNg sequence was obtained from a cDNA fragment produced from mRNA of human peripheral blood lymphocytes activated by mitogen agents;
   the late polyadenylation site from bovine growth hormone (BGHpolyA) which ensures the termination of transcription;
the remaining part of the adenoviral genome from nucleotide 3512 to the right end including the deletion of the E3 region.

ASN-002 requires storage at −80° C. due to its limited stability at higher temperatures.

### R150-400 Depot

The pH of a water and tetraethyl orthosilicate (TEOS) mixture at an initial molar ratio of 150:1 was adjusted to pH 2 with hydrochloric acid and vigorously stirred at room temperature for 25 min. The pH of the sol was adjusted to the desired pH (6, 6.5 or 7) by adding 0.1 M NaOH. The sol was cooled in an ice-water bath and the desired amount of ASN-002 is added (1.5×10¹¹ vp/ml). The sol was diluted with water so that the final water:TEOS ratio is 400:1. Placebo microparticles (see below) are added to the sol in a ratio 0.5 g particles per 1 ml. The suspension can be allowed to gel and used to fill syringes or the syringes can be filled with the suspension and allowed to gel in a rotator (3 days 24° C. and 9 days at 4° C.).

### R5-400 Depot

This depot preparation was made as R150-400 above but with water and TEOS in a molar ratio 5:1. Suspensions were allowed to gel as above for 12 days at 4° C.

### Placebo (“Secondary”) R5 Microparticles

The R5 sol was made using water:TEOS at a ratio of 5:1 with HCl as catalyst (pH 2). The sol was diluted with ethanol, and pH is adjusted to 6.3. The diluted sol was spray dried using GeaMinor mobile minor spray dryer.

### R400 Hydrogel

R400 hydrogel was made using water:TEOS at a ratio of 400:1 and pH adjusted to 2 with HCl as above. The pH was adjusted to 6, 6.5 or 7 with 0.1M NaOH. The mixture was cooled in an ice-water bath, and ASN-002 added to the required concentration.

### R100-400 Depot

This depot preparation was made as R150-400 above but with water and TEOS an initial molar ratio 100:1 and pH adjusted to 6 with 0.1 M NaOH. The final virus content was 1.5×10¹¹ vp/ml.

### Analytical Assays

The number of viral particles in ASN-002 or the various formulations was determined using an HPLC assay. Ion exchange-HPLC analysis was performed with bioinert Agitent 1260 infinity II uHPLC using a 500 μl injection volume. (Ball et al. 2010: Rapid Analysis of Adenoviruse Type 5 Particles with Bio-Monilith Anion-Exchange HPLC Columns).

The ability of ASN-002 to transduce host cells and induce expression of IFN-γ was measured by assaying IFN-γ secretion from transduced H-1299 cells in a quantitative cytokine ELISA. Briefly, H-1299 cells were cultured in a constant number in a 96-well cell culture plate (50,000 cells/well) and serial dilutions made from the ASN-002 or ASN-002 formulations and added onto the cells. All dilutions were calculated as a theoretical viral particle count per cell number. After a 24 h infection period, the culture wells were drained and washed with PBS, fresh culture medium was added, and the culture plates were incubated in a 37° C. cell culture incubator for 24 hours. Supernatants were collected, and the concentration of IFN-γ produced by infected cells in 24 hours was measured by ELISA.

The infectivity and ability of ASN-002 to form viable viral particles were measured using REK293T cells. The infectivity was compared using TCID50 assays.

### Results

### Stability of ASN-002 Alone and in SiO₂ Formulations Following Pre-Incubation at 37° C. and 4° C.

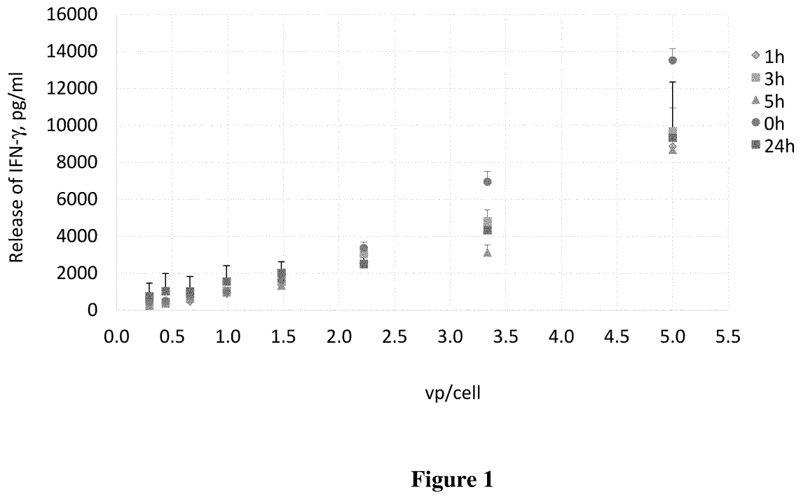

After an incubation period of ASN-002 virus (3×10⁹ vp/ml) of even one hour at 37° C. and up to 24 hours before infection of host H-1299 cells, a substantial amount of viral activity, as assessed by IFN-γ release, was lost relative to virus not incubated at 37° C. prior to infection (FIG. 1). Thus, ASN-002 rapidly loses activity upon exposure to a physiological temperature.

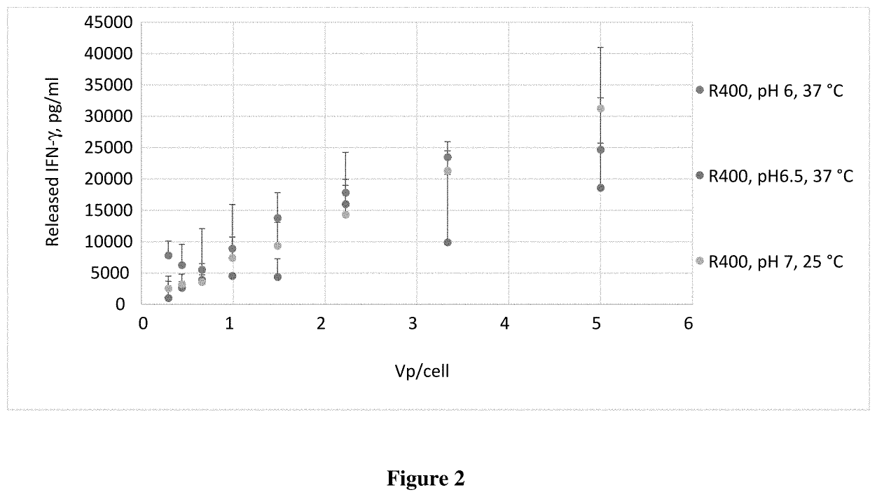

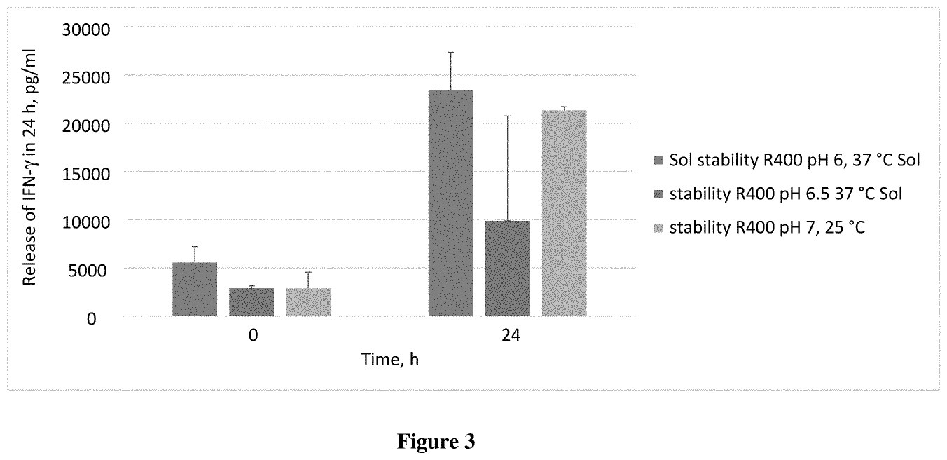

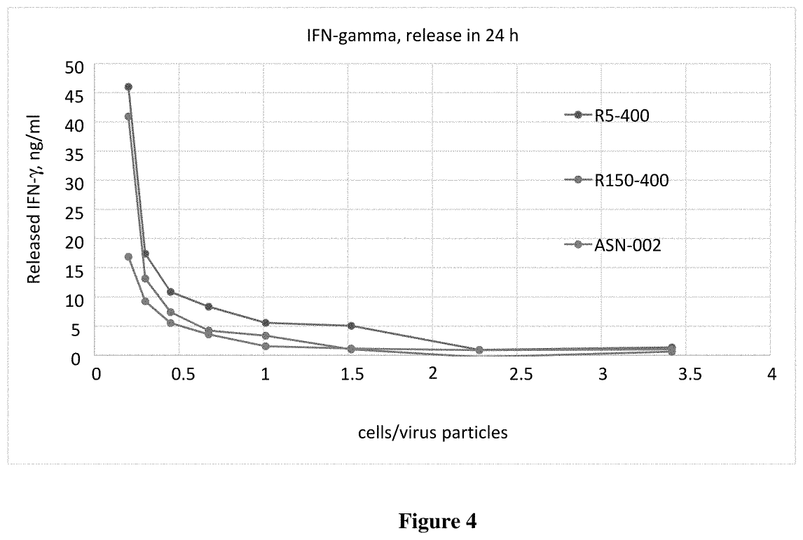

In order to determine whether ASN-002 could be stabilized at elevated temperatures, it was formulated in an R400 sol made at different pHs and incubated at 37° C. for 24 h. Samples were diluted to various vp/cell and added to H-1299 cells. IFN-γ release was measured as described previously. As shown in FIG. 2, there were no significant differences between the various R400 sol preparations, but each of these appeared to be more active than non-formulated virus (compare to FIG. 1 and see FIG. 4). In the R-400 ASN-002 formulations, it was observed that pre-incubation at 37° C. resulted in greater activity than the same formulation with no 37° C. incubation prior to infection (FIG. 3).

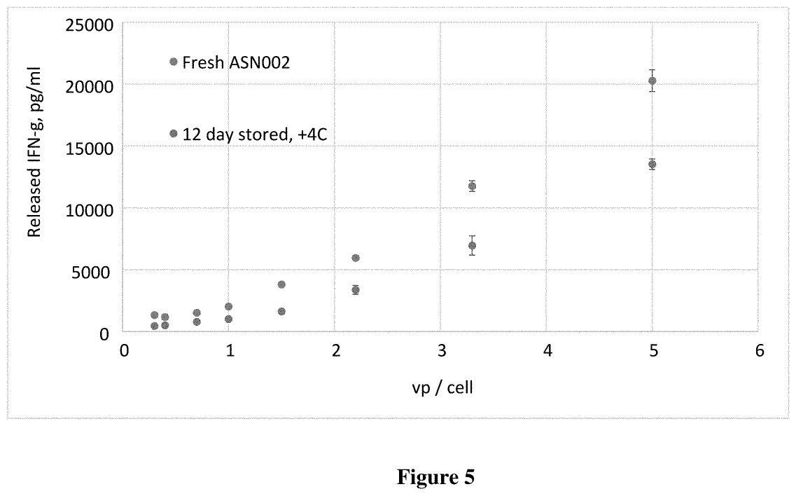

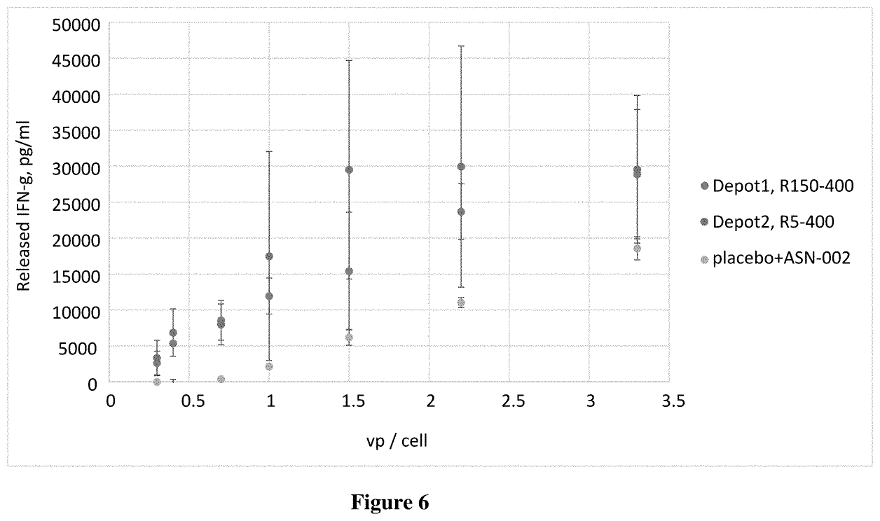

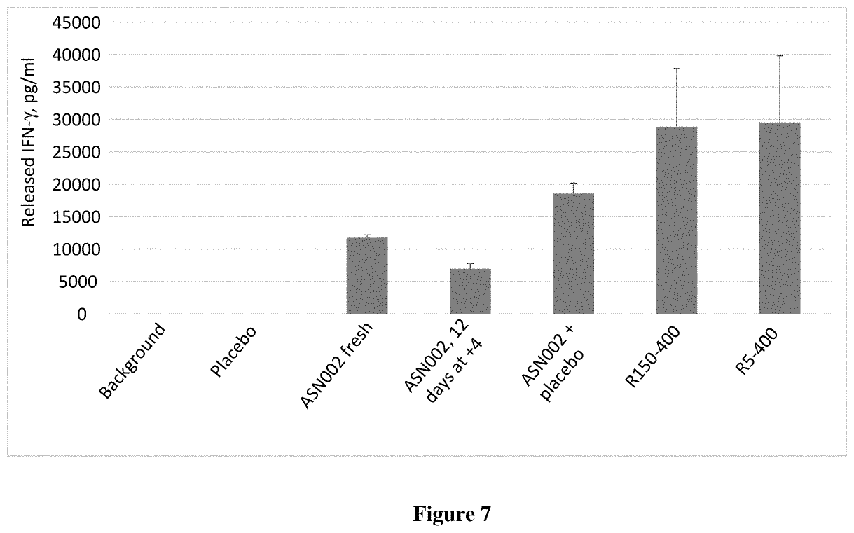

In order to assess the long-term stability of unformulated versus formulated. ASN-002, the inventors compared the infectivity of freshly thawed ASN-002 versus ASN-002 stored at 4° C. for 12 days. As shown in FIG. 5, ASN-002 lost a substantial amount of activity over the 12 day incubation period. The inventors subsequently assessed whether R150-400 and R5-400 formulations of ASN-002 would preserve ASN-002 activitity over prolonged storage periods at 4° C. As shown in FIG. 6, the ASN-002 activity of both of these formulations following 4° C. storage for 7 days was substantially higher than that observed for a control preparation in which ASN-002 was mixed (not encapsulated) with silica microparticles. As summarised in FIG. 7, there is an increase viral activity when ASN-002 is formulated as R150-400 and R5-400. The increased activity is observed relative to both freshly thawed (unformulated) ASN-002 and pre-incubated (4° C.) unformulated ASN-002. Interestingly, there was a slight increase in ASN-002 activity observed even in the ASN-002+Si-microparticles control preparation (“ASN-002+placebo”).

### Dissolution Characteristics of the ASN-002 R150-400 Formulation

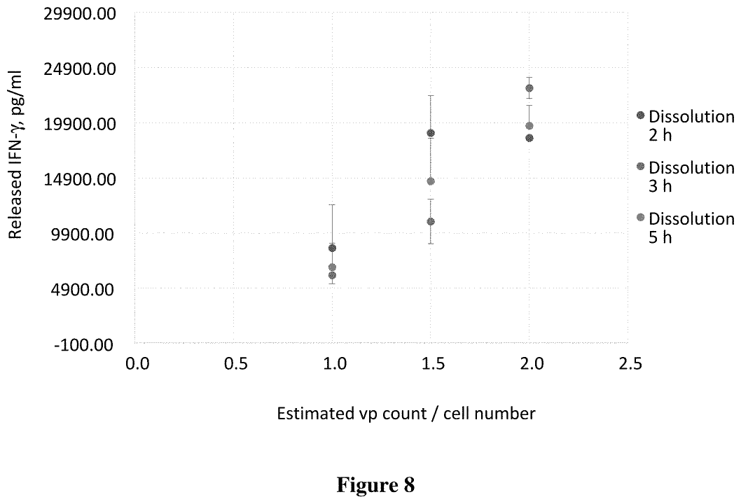

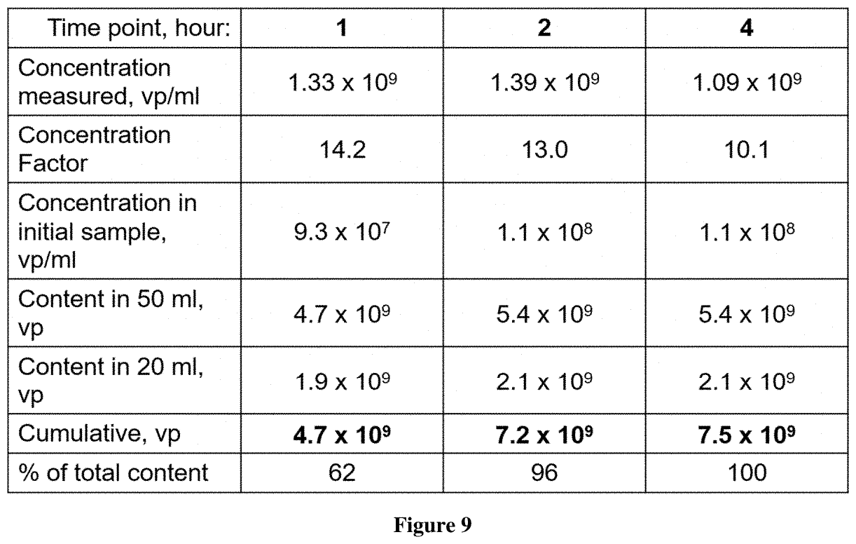

In a follow-up experiment, the dissolution of the ASN-002 R150-400 formulation was assessed as a function of time and estimated viral particle (“vp count”/cell number). Cells were infected with culture medium in which the ASN-002 R150-400 formulation was allowed to dissolve for 2, 3 and 5 hours. As shown in FIG. 8, viral release appeared to plateau over a 2-5 hour period. In order to more directly assess viral particle release in a buffered Tris solution was determined at 1, 2 and four hours as described above. As summarised in FIG. 9, approximately 62%, 96% and 100% of the viral particles were released at 1, 2 and 4 hours, respectively.

The above data show that SiO₂-gel matrix-based formulations preserve ASN-002 activity over prolonged periods of time at elevated temperatures ranging from 4° C. to 37° C. In addition, these formulations enhance the infectivity of ASN-002 relative to unformulated ASN-002, Thus, SiO₂-gel matrix-based formulations of, offer considerable advantages for applications requiring controlled and extended release of ASN-002, and other recombinant viruses, particularly in vivo, e.g., for combination therapy cancer treatments.

### Infectivity and IFNγ Secretion by Virus Formulated in R100-400 Depot Formulation

Analysis of virus particle number (3×10¹¹) and the TCID50 (6.7×10⁹ TCID₅₀/ml) determined in the HEK293T infection assasy of ASN-002 reference standards stored at −80° C. indicated a vp/TCID50 ratio of 45 (a variation of 38-75). To quantitate the extent of virus release, infection and expression of IFNγ a sample of 100 ul of R100-400 depot formulation with 1.5×10¹⁰ vp was subjected to a dissolution in 50 ml Tris-Tween buffer (50 mM Tris, pH 7.4, 0.01% Tween 80) at RT. Based on a vp/TCID₅₀ of 45 the total calculated infective titre is 3.3×10⁸ TCID. Samples were analysed for vp and TCID₅₀ at 5, 12 and 24 hr.

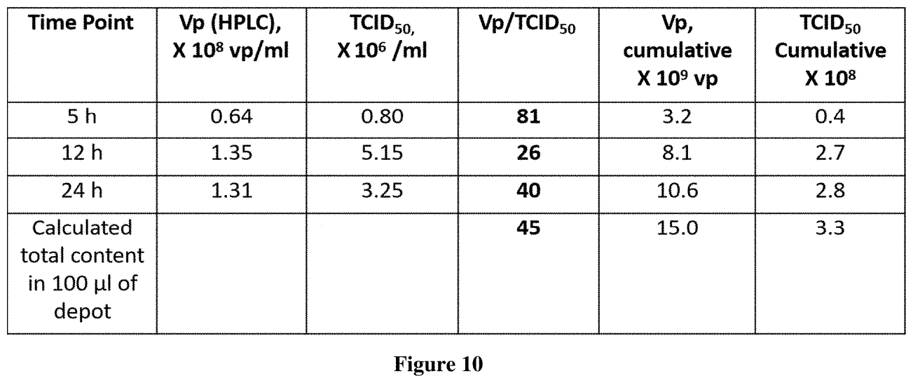

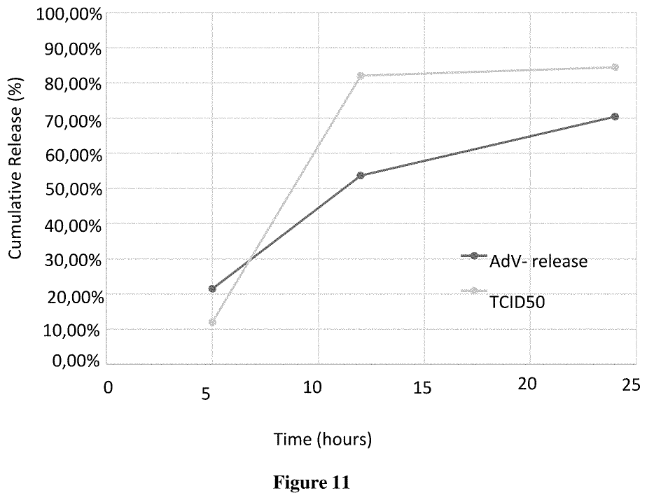

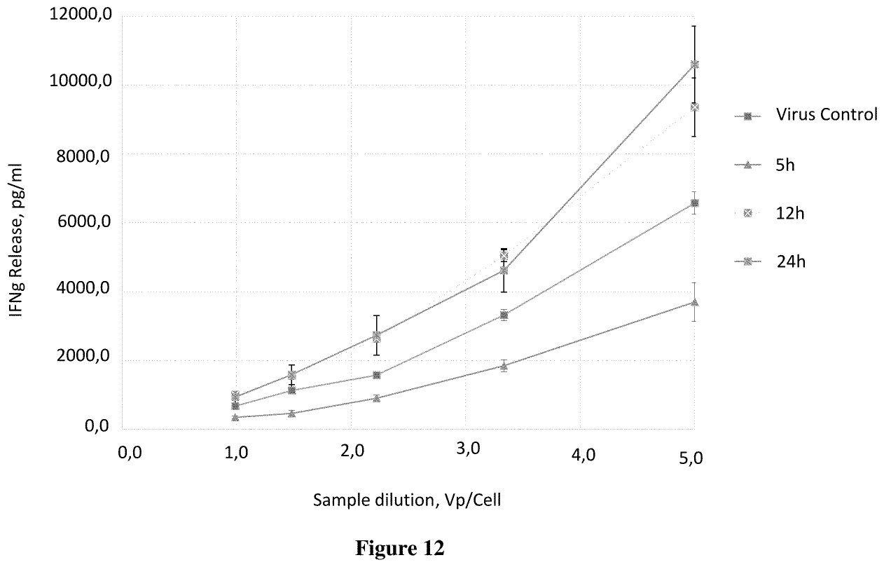

Based on the viral particle count and infection assay it is apparent that greater than 80% of activity and >70% of vp are recovered after the 24 hr period (FIGS. 10 and 11). Similarly the IFNγ expression assay at 12 and 24 hr the level of IFNγ is approching a maximum. A reference unformulated ASN-002 control was also used in the expression assay and FIG. 12 clearly indicates that the ASN-002 released from the depot formulation generates more IFNγ than the unformulated ASN-002.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU2018900204 filed 23 Jan. 2018, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Accart et al. (2013) J Transl Med. 11:226.
Batra et. al. (1999) Am. J. Respir. Cell Mol. Biol. 21:238-245.
Bett et al. (1993) J. Viral. 67:5911-5921.
Bhatia et al. (2016) Oncolytic Virotherapy 5:99-113.
Blackwell et al. (1999) Arch. Otolaryngol. Head, Neck Surg. 125:856-863.
Chodorge et al. (2012) Cell Death & Differentiation 19:1187-1195.
Dorai et al. (1999) Int. J. Cancer 82:846-852.
Dreno et al. (2014) PLoS One 9:e83670.
Dummer et al. (2004) Blood. 104:1631-1638.
Dummer et al. (2010) Mol Ther. 18:1244-1247.
Ebert et al. (2007) Nature Methods 4:721-726,
Feldman et al. (1996) Cardiovasc. Res. 32:194-207.
Guo et al. (2002) Blood 99:3419-3426.
Han et al. (1999) Biol. Pharm. Bull. 22:836-840.
Hermens and Verhaagen (1998) Prog. Neurobiol. 55:399-432.
Hillman et al. (2004) Cancer Gene Therapy 11:61-72.
Inci et al. (1999) Antisense Nucleic Acid Drug Dev. 9:341-349.
Khammari et al. (2015) Cancer Immunol Immunother 64:805-815.
Lesch (1999) Biol. Psychiatry 45:247-253.
Liu et al. (2004) PNAS USA 101 Suppl 2:14567-14571.
Liu et al. (2011) Postgrad Med J. 87:487-495.
Marienfeld et al. (1999) Gene Then 6:1101-1113.
Martinez-Sales (1999) Current Opinion in Biotechnology 10:458-464.
McElrath et al. (2008) Lancet 372:1894-1905.
Mincheff et al. (2000) Eur. Ural. 38:208-217.
Parks et al. (1997) J. Viral. 71:3293-3298.
Petrof (1998) Eur. Respir. J. 11:492-497.
Rosenfield et al. (1992) Cell 68:143-155.
Stewart et al. (1999) Gene Ther. 6:350-363.
Trichas et al. (2008) BMC Biology 6:40.
Urosevic (2007) Curr Opin investig Drugs 8:493-498.
Vanderkwaak and Alvarez (1999) Curr. Opin. Obstet. Gynecol. 11:29-34.
Volpers and Kochanek (2004) The Journal of Gene Medicine 6:S164-S171.
Wilson (1996) Clin. Invest. 98:2435.
Yamamoto et al. (2017) Cancer Sci. 108:831-837.
Yoon et al. (2016) Expert Opin Drug Deity. 13:843-858.

1. A pharmaceutical composition comprising:
(i) one or more non-replicative recombinant adenoviruses for expression of one or more biotherapeutic agents; and
(ii) SiO₂ matrix hydrogel;
wherein the one or more non-replicative recombinant adenoviruses are interspersed in the SiO₂ matrix hydrogel, and wherein the pharmaceutical composition does not comprise a chemotherapeutic agent.

2. The pharmaceutical composition according to claim 1, wherein a therapeutically effective dose of the one or more non-replicative recombinant adenoviruses in the pharmaceutical composition is lower than a therapeutically effective dose of the same non-replicative recombinant adenoviruses not formulated in the pharmaceutical composition.

3. The pharmaceutical composition according to claim 1, wherein the one or more biotherapeutic agents are selected from the group consisting of: cytokines, chemokine, chemokine agonist, chemokine antagonist, chemokine receptor antagonist, costimulatory molecules, checkpoint inhibitors, metalloproteinase inhibitors, matrix metalloproteinase (MMPs) inhibitors, tissue inhibitors of metalloproteinases (TIMPs) and antibodies.

4-5. (canceled)

6. The pharmaceutical composition according to claim 1, wherein at least one of the biotherapeutic agents is a cytokine.

7. The pharmaceutical composition according to claim 6, wherein the cytokine is interferon gamma.

8. The pharmaceutical composition according to claim 7, wherein one of the one or more non-replicative recombinant adenoviruses is ASN-002.

9. The pharmaceutical composition according to claim 1, wherein at least one of the biotherapeutic agents is a CD40L or an CD27 agonist.

10. The pharmaceutical composition according to claim 1, wherein expression of at least one of the biotherapeutic agents is higher in vivo when compared to a corresponding pharmaceutical composition lacking the SiO₂ matrix hydrogel.

11. The pharmaceutical composition according to claim 1, wherein the one or more non-replicative recombinant adenoviruses comprise a first and a second non-replicative recombinant adenoviruses each of which is for expression of a different biotherapeutic agent.

12. The pharmaceutical composition according to claim 1, wherein the SiO₂ matrix hydrogel comprises water and tetraethyl orthosilicate (TEOS) in a final molar ratio of between about 5:1 to about 4,000:1.

13. (canceled)

14. The pharmaceutical composition according to claim 1, further comprising one or more pharmaceutically acceptable excipients.

15-26. (canceled)

27. The pharmaceutical composition according to claim 1, wherein the one or more non-replicative recombinant adenoviruses retain at least about 50% to about 75% of their infectivity when the pharmaceutical composition is maintained at about 4° C. for about 12 months to about 24 months.

28. The pharmaceutical composition according to claim 1, wherein the pharmaceutical composition is a depot formulation.

29. The pharmaceutical composition according to claim 1, wherein the pharmaceutical composition comprises about 1×10¹⁰ viral particles/ml to about 5×10¹² viral particles/ml.

30. A method for treating a subject suffering from a disease, the method comprising administering to the subject a therapeutically effective amount of the pharmaceutical composition according to claim 1.

31. The method according to claim 30, wherein the disease is cancer.

32. The method according to claim 31, wherein the cancer is selected from the group consisting of basal cell carcinoma, squamous cell carcinoma, colorectal cancer, ovarian cancer, breast cancer, gastric cancer and pancreatic cancer.

33. The method according to claim 31, wherein the cancer comprises one or more lesions or tumours.

34. The method according to claim 33, wherein the pharmaceutical composition is injected into at least one of the one or more lesions or tumours.

35. The method according to claim 32, wherein the cancer is a basal cell carcinoma or squamous cell carcinoma.

36-39. (canceled)
